(19) United States
(12) Patent Application Publication
(10) Pub. No.: US 2022/0334134 A1
(43) Pub. Date: Oct. 20, 2022

(54) CLEAN SCREEN CONTACTLESS ANALYTE DETECTION TEST

(71) Applicant: DaVinci Plastic Surgery, LLC, Washington, DC (US)

(72) Inventors: Steven DAVISON, Washington, DC (US); Katherine Lynn-Long LIJOI, Arlington, VA (US)

(21) Appl. No.: 17/723,112

(22) Filed: Apr. 18, 2022

Related U.S. Application Data
(60) Provisional application No. 63/177,239, filed on Apr. 20, 2021.

(51) Int. Cl. G01N 33/76; G01N 33/543

(52) U.S. Cl. CPC G01N 33/76; G01N 33/54391; G01N 2333/59

(57) ABSTRACT

The invention encompasses a method and a device for a contactless pregnancy detection including a conical shaped main body including a first end having a first opening and a second end having a second opening, a longitudinal crease allowing the conical body to lay flat when not in use, including a pregnancy detection element mounted at an edge of the second opening, and a flap extending from the conical body to allow a user to hold the device.

### FIELD OF THE INVENTION

The invention encompasses a method and a device for a contactless detection of an analyte including a device comprising a conical shaped main body including a first end having a first opening and a second end having a second opening, an optional first and second longitudinal crease opposite from one another to allow the conical body to lay flat when not in use, including a detection element mounted proximate to the second opening, and an extension from the conical body to allow a user to hold the device.

### BACKGROUND OF THE INVENTION

Surgical procedures rely heavily on investigative testing performed on various compositions of biological material prior to surgery to ensure the safety of a subject and determination of whether the subject is pregnant. Testing liquid samples, such as bodily fluids, often involves collecting a sample of a liquid and exposing the liquid to a diagnostic testing assay. An assay generally involves bringing a portion of the liquid sample into contact with an assay or test strip in order to qualitatively assess or measure the presence or amount of the functional activity of an anticipated analyte. Many such test assays or strips are known to detect for the presence of a specific chemical, hormone, or other material in liquid, such as blood, urine, or saliva. Such tests have been designed, manufactured, produced, and marketed for use in such fields as home, industrial, veterinary, surgical, and occupational testing, among others.

Test devices are available which allow a user to test for the presence of, for example, human chorionic gonadotropin (HCG), an early indicator of pregnancy, in urine. Devices and assays have also been created for the detection of a variety of other chemicals in urine, including screenings for illegal drugs in a test subject in, for example, employment or correctional settings. These devices, to date, require that a user or test subject either urinate directly on the device or assay or to deposit urine into a receptacle into which a testing assay or strip is placed. Unfortunately, due to the exposure of both the receptacle and the assay to the test subject, support staff, lab personnel, and even the atmosphere, contamination of the sample and/or the assay is possible even when the utmost care is observed during all steps of these testing processes. Accordingly, a need for a test device which reduces the potential opportunity for contamination of an assay or test strip is needed.

### SUMMARY OF THE INVENTION

The invention encompasses a method and a device for a contactless detection of an analyte in a body fluid sample, the device including a conical shaped main body including a first end having a first opening and a second end having a second opening, a first longitudinal crease and second longitudinal crease allowing the conical body to lay flat when not in use, including an analyte detection element mounted at an edge of the second opening, and a flap extending from the conical body to allow a user to hold the device.

The invention encompasses a device for detection of a human or veterinary analyte the device comprising: a conical main body comprising a first end and a second end, wherein the first end comprises a first wider opening and the second opening comprises a narrower end comprising a second opening positioned opposite to the first opening, wherein the conical body includes an extended portion for holding the device, and wherein the conical body includes a detection element to detect an analyte of interest, wherein the detection element is mounted closer to the second opening.

In certain embodiments, the conical device includes a first longitudinal crease and a second longitudinal crease, wherein the first longitudinal crease and the second longitudinal crease are capable of being folded to have a compact flattened effect, for example for packaging of the device, wherein the device is opened by applying simultaneous pressure to the first longitudinal crease and the second longitudinal crease, wherein when in operation.

In certain embodiments, the first opening of the first end is configured to receive an analyte from a subject while the subject is in a standing position and enables the analyte to contact the detection element at the second opening, and wherein when the analyte comes in contact with the detection element, the detection element detects the presence of analyte and indicates a positive or negative output if the analyte is present in the body fluid sample.

In certain embodiments, the invention encompasses a device for a contactless pregnancy detection including a conical shaped main body including a first end having a first opening and a second narrower end having a second opening, a first longitudinal crease and a second longitudinal crease opposite the first crease allow the conical body to lay flat when not in use including a pregnancy detection element mounted closer to the second opening and a flap extending from the first end of the conical body to allow a user to hold the device.

In certain embodiments, the invention encompasses a device for detection of a human analyte, the device comprising: a conical main body comprising a first end and a second end, wherein the first end comprises a first opening and the second end comprises a second opening positioned opposite to the first opening in a narrowing manner, a pregnancy detection element that is mounted proximate to the second opening; and an first longitudinal crease and a second longitudinal crease opposite the first crease, wherein the first longitudinal crease and the second longitudinal crease are capable of being folded to flatten the device, wherein the device is opened by applying simultaneous pressure to the first end of the first longitudinal crease and the second longitudinal crease, further comprising an extension proximate the first end to allow a subject to hold the device, wherein the first opening of the first end is configured to receive urine from a female user while the female user is in a standing position and enables the urine to pass through a pregnancy detection element proximate the second opening, and wherein when the urine comes in contact with the pregnancy detection element, the pregnancy detection element immunologically detects human chorionic gonadotropin (hCG) present in the urine of the female user and indicates a positive pregnancy result if the female user is pregnant.

Although the following detailed description contains many specific details for the purposes of illustration, anyone of ordinary skill in the art will appreciate that many variations and alterations to the following details are within the scope of the invention. Accordingly, the exemplary embodiments of the invention described below are set forth without any loss of generality to, and without imposing limitations upon, the claimed invention.

### BRIEF DESCRIPTION OF THE DRAWINGS

The embodiments herein will be better understood from the following detailed description with reference to the illustrative exemplary drawings.

FIG. 1. illustrates a top view of a device for pregnancy detection, according to an embodiment herein.

FIG. 2. illustrates a side view of a device for pregnancy detection, according to an embodiment herein.

### DETAILED DESCRIPTION OF THE INVENTION

In its broadest embodiment, the invention encompasses a method and a device for a contactless detection of an analyte in a body fluid sample, the device including a conical shaped main body including a first end having a first opening and a second end having a second opening, a first longitudinal crease and second longitudinal crease allowing the conical body to lay flat when not in use, including an analyte detection element mounted at an edge of the second opening, and a flap extending from the conical body to allow a user to hold the device.

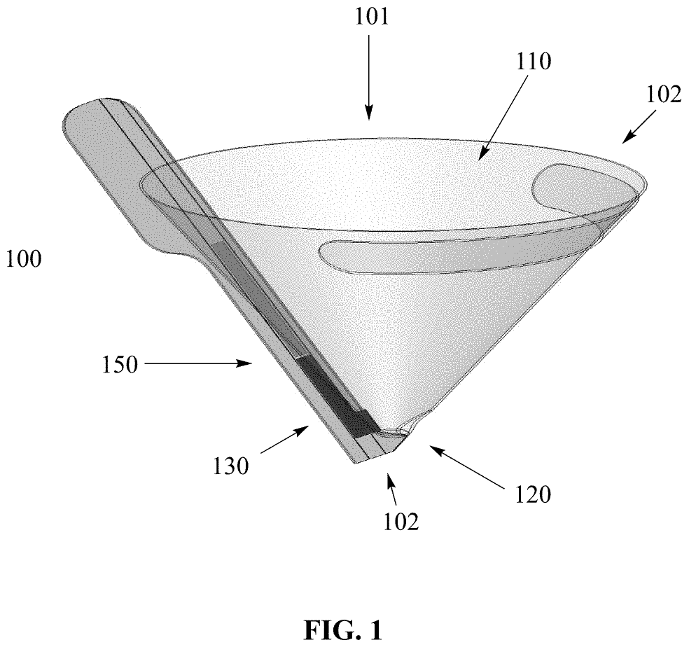

Referring now to FIG. 1 of the drawings, there is shown a device 100 to detect the presence of a specific analyte in a test sample of a fluid, for example, bodily fluid, preferably urine. A visual indication is provided to a user or other analyst of the test sample, which represents the positive or negative presence of the analyte in the test sample. A control indicator is also provided to visually assure the user or other analyst of the validity of the assay or test result. In certain embodiments, the test device 100 can detect the presence of an analyte, antigen, ligand, or macromolecule in fluids such as bodily fluids, including but not limited to urine, blood, saliva, mucus, or perspiration. In certain embodiments, the biologic fluids used for testing include urine, saliva, cerebrospinal fluid (CSF), amniotic fluid, synovial fluid, pleural fluid, peritoneal fluid and pericardial fluid. These fluids often contain the same biologic analytes of interest — such as glucose and protein — but differ greatly from each other in physical and chemical properties. In certain embodiments, the biological fluids can be further diluted with a solvent for with the device and methods of the invention.

The visual indicators use a reagent which reacts to the presence of the analyte and is a conjugate of reactive color particles and/or an antibody specific to the anticipated analyte.

Device 100 is for example an antigen/receptor assay, which can be used to detect the presence of an analyte in a test fluid such as urine. In certain embodiments, the devices are used in home, industrial, veterinary, surgical, and occupational testing centers among others. Generally, devices such as these are found in home testing or clinical testing devices, for example, pregnancy detection systems, disease detection systems, or apparatus for the detection of illegal drugs. Such assays may involve an antigen/antibody reaction wherein a synthetic conjugate provides a visible tag. A specific antibody is provided specific to the antigen being detected. An absorbable indicator is provided, generally providing a simple positive/negative indicator for the presence of the antigen being detected. A control indicator is also provided to ensure the validity of the test result. Many detection elements are commercially available and could be used to test for a plurality of anticipated analytes in accordance with the embodiments described herein.

Test device 100, in accordance with the present invention, is a device for collecting and analyzing a sample such as, for example, a bodily fluid. The device generally includes a conical shape having a first opening 110 which provides a means for collecting the body fluid and a second opening 120, which allows liquid to exit the conical device. In certain embodiments, a typical conical container 100 may have a capacity of between about 100 to about 500 mL; however, the present invention is not intended to be limited to this size or volume.

The conical device may be formed, or molded, from any suitable material, such as plastic or paper. In certain embodiments of the invention, conical device is constructed of a transparent material such that the visual indications provided by a completed assay are visible through the surfaces of the container.

As shown in the example of FIG. 1, a test strip 130 is affixed proximate to the second opening on the interior surface of conical device. In alternative embodiments, the test strip 130 may be affixed to an inside surface of the wall of conical device proximate the second narrow opening. The test strip 130 may be designed to test for the presence of a specific analyte, and it should be appreciated that alternative embodiments of the present invention allow for any number of analyte specific tests be performed with the device 100 of the invention. In the present example below, the test strip 130 is configured to test for the presence of, for example, Human Chorionic Gonadotropin (HCG), a hormone produced by a portion of the placenta following implantation of a blastocyst in human females during embryogenesis; the presence of this hormone in urine is an early indicator of pregnancy.

In order to avoid contamination of test strip 130, in certain embodiments an optional removable protective strip is secured in place over test strip 130 at the time device 100 is manufactured or when test strip 130 is affixed to container 100. Alternative embodiments provide that the test strip 130 and protective strip comprise a replaceable “test panel” which can be loaded or fastened into an appropriately sterilized device 100. Protective strip prevents competing analytes or other contaminants from coming into contact with test strip 130 during the time period between manufacture/assembly and use. Such contaminants include but are not limited to microbes and other atmospheric contaminants, and contaminants present on a user or analysts skin including hormones, microbes, oils, or perspiration. Protective strips may be composed of any number of materials appropriate to preventing exposure of the test strip 130 to atmospheric or other contaminants, including but not limited to plastic, metal foil, or an adhesive coating. In certain embodiments, the protective strip is a coating which is dissolvable in the desired test liquid, and accordingly, the protective strip dissolves after a certain period of exposure to the test liquid, allowing the test strip 130 to be exposed to the test liquid. In alternative embodiments, an adhesive is applied to the area surrounding the test strip 130, and the protective strip is secured in place around and over (respectively) the test strip 130, preventing the test strip 130 from being exposed to atmospheric or other contaminants.

In certain embodiments of the invention, once the protective strip is removed from contact with the test strip 130, the test strip 130 will be immediately exposed to the desired sample of test liquid present in the device 100 thereby contacting the minimum potential intermediary contaminants. Once the protective strip is removed, the assay configured on the test strip 130 will develop accordingly, allowing a user or other analyst to determine whether or not an anticipated analyte is present in the test liquid via the visual indicator provided by the completed assay.

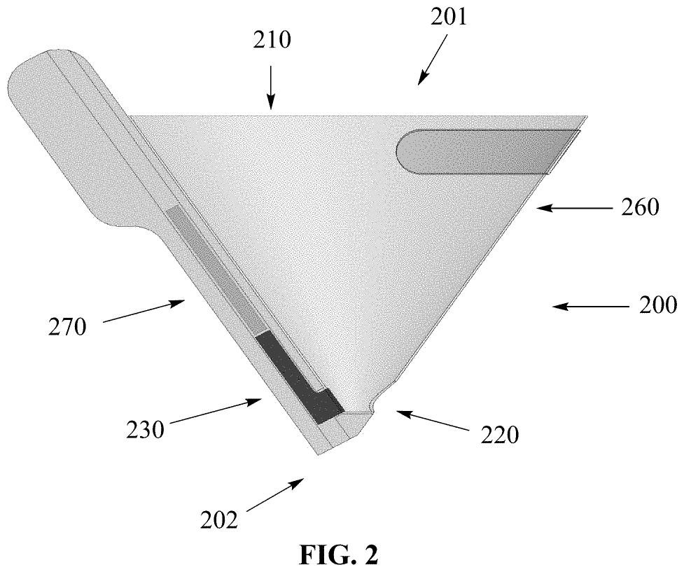

In FIG. 2, there is shown an exemplary, illustrative testing device 200 for conducting an immunoassay to detect the presence of a specific analyte in a test sample of test fluid, for example, bodily fluid. A visual indication is provided to a user or other analyst of the test sample which represents the positive or negative presence of the analyte in the test sample. A control indicator is also provided to visually assure a user or other analyst of the validity of the assay or test result. The device 200 is capable of detecting the presence of an analyte, antigen, ligand, or macromolecule in fluids such as bodily fluids, including but not limited to urine, blood, saliva, mucus, or perspiration. The visual indicators use a reagent which reacts to the presence of the analyte and is a conjugate of reactive color particles and/or an antibody specific to the anticipated analyte.

The system 200 may utilize, for example, an antigen/receptor assay device which can be used to detect the presence of an anticipated, analytes, in a test fluid such as urine. As provided above, such devices may be utilized in home, industrial, veterinary, surgery, and occupational testing, among others. Generally, devices such as these are found in clinical testing systems, for example, pregnancy detection systems, disease detection systems, or apparatus for the detection of illegal drugs. Such assays may involve an antigen/antibody reaction wherein a synthetic conjugate provides a visible tag. A specific antibody is provided specific to the antigen being detected. An absorbable indicator is provided, generally providing a simple positive/negative indicator for the presence of the antigen being detected. A control indicator is also provided to ensure the validity of the test result. Many test strips are commercially available and could be used to test for a plurality of anticipated analytes in accordance with the embodiments described herein.

In another embodiment, a pregnancy detection device is coated with a plurality of human chorionic gonadotropin (hCG) antibodies for detecting the human chorionic gonadotropin (hCG) present in the urine of the female user. In another embodiment, the pregnancy detection element is capable of testing and indicating the positive pregnancy result, or a negative pregnancy result, or a failed result.

In yet another embodiment, the pregnancy detection element operates by immunologically detecting a plurality of morphological differences between the human chorionic gonadotropin (hCG) and modified forms thereof, which are secreted into the urine of a pregnant female. In yet another embodiment, the first opening of the first end is configured to enable the female user to urinate while in standing position.

In yet another embodiment, the pregnancy detection element tests and starts indicating the positive pregnancy result, or the negative pregnancy result or the failed result within 15 seconds of coming in contact with the urine of the female user.

In yet another embodiment, the device is made from a suitable material selected from a group comprising a water-absorbent paper, a cardboard, a water dissolvable plastic-like film, and the like.

In yet another embodiment, the device is biodegradable and environment friendly after being used by the female user. In yet another embodiment, the second opening of the second end is configured to discharge urine from the female user after the urine has come in contact with the pregnancy detection element. In yet another embodiment, a perimeter of the first opening is greater than a perimeter of the second opening.

In yet another embodiment, the first opening comprises a plurality of walls defined through the upper longitudinal crease and the lower longitudinal crease, wherein the plurality of walls comprises inner surfaces coated with a thin, moisture-resistant coating for preventing the urine from passing through the plurality of walls. In yet another embodiment, the device is capable of conducting a plurality of urine related tests including but not limited to a urinary tract infection (UTI) test, and the like.

In another aspect of the present disclosure, a method for pregnancy detection for detecting pregnancy using urine from the female is provided, the method comprising the steps (a) providing a pregnancy detection device as described herein to a female user, (b) receiving urine from the female user into a first opening of a first end of the pregnancy detection device, (c) flowing urine from the female user from the first opening towards a pregnancy detection element mounted at an edge of a second opening of a second end of the pregnancy detection device, (d) testing and start indicating a positive pregnancy result, or a negative pregnancy result, or a failed result by the pregnancy detection element within 15 seconds of coming in contact with urine of the female user, and (e) freely discharging urine from the female user via the second opening after the urine has come in contact with the pregnancy detection element.

In yet another aspect of the invention, a device for pregnancy detection is provided, the device comprising: a conical main body comprising a first end and a second end, wherein the first end comprising a first opening and the second end comprising a second opening positioned opposite to the first opening, wherein a perimeter of the first opening is greater than a perimeter of the second opening; a pregnancy detection element that is mounted inside the device proximate to the second opening; and an first longitudinal crease and a second longitudinal crease opposite the first longitudinal crease, wherein the first longitudinal crease and the second longitudinal crease are folded to have a flattened packaging of the device, wherein the device is opened by applying simultaneous pressure to the first longitudinal crease and the second longitudinal crease, wherein, when in operation, the first opening of the first end is configured to receive the urine from a female user while the female user is in a standing position and enables the urine to pass through the pregnancy detection element at the edge of the second opening, wherein when the urine comes in contact with the pregnancy detection element, the pregnancy detection element immunologically detects human chorionic gonadotropin (hCG) present in the urine of the female user and indicates a positive pregnancy result if the female user is pregnant.

In certain embodiments, the pregnancy detection device is coated with a plurality of human chorionic gonadotropin (hCG) antibodies for detecting the human chorionic gonadotropin (hCG) present in the urine of the female user. In another embodiment, the second opening of second end is configured to discharge the urine from the female user after the urine has come in contact with the pregnancy detection element.

In yet another embodiment, the pregnancy detection element tests and starts indicating the positive pregnancy result, or a negative pregnancy result or a failed result within 15 seconds of coming in contact with the urine of the female user. In yet another embodiment, the device is biodegradable and environment friendly after being used by the female user. In yet another embodiment, the device is made from a suitable material selected from a group comprising a water-absorbent paper, a cardboard, a water dissolvable plastic-like film, and the like.

Those of ordinary skills in the art will appreciate that embodiments of the disclosure disclose a modern pregnancy device that replaces the traditional way to test the pregnancy. The device provides the comfort to expecting women to check their pregnancy with ease, speed and hygiene. Essentially, the device as disclosed herein below is a funnel or a vessel or any other suitable shape, which indicates the pregnancy status. It does not require any dropper or cup to store urine and test the pregnancy. The entire process is hygienic, and user does not come in contact with urine and need not sit on the toilet.

In other embodiments, the disclosure comprises a device for selectively or preferentially detecting hyper glycosylated human chorionic gonadotropin (hCG-H), in the female subject's urine without causing the user to come into contact with the liquid waste.

FIG. 1. illustrates a top view of a device 100 for pregnancy device, according to an embodiment herein. The device 100 for pregnancy detection includes, a conical main body 102 including a first end 101 and a second end 102. The first end 101 includes a first opening 110 and the second end 102 includes a second opening 120 positioned opposite to the first opening 110.

In accordance with an embodiment of the disclosure, a perimeter of the first opening 110 is greater than a perimeter of the second opening 120.

In accordance with an embodiment of the present disclosure, the device 100 further includes a pregnancy detection element 130 that is mounted at an edge of the second opening 120. In use, the pregnancy detection element 130 tests the urine and indicates a positive pregnancy result, or a negative pregnancy result, or a failed result. In operation, the pregnancy detection element 150 includes a test strip 130 operates by immunologically detecting a plurality of morphological differences between human chorionic gonadotropin (hCG) and modified forms thereof, which are secreted into the body fluid (e.g. urine) of a pregnant female. Optionally, the pregnancy detection element includes a control strip and the test strip 130.

In accordance with an embodiment of the present disclosure, the first opening 101 of the first end 110 is configured to receive urine from a female user. In use, the first opening 101 of the first end 110 is configured to allow the female user to urinate while in standing position by slightly tilting (bending) her hips in a manner such that the device 100 is tilted downwards.

In accordance with an embodiment of the present disclosure, the pregnancy detection element 130 tests and starts indicating the positive pregnancy result, or the negative pregnancy result or the failed result within 15 seconds of coming in contact with urine of the female user.

FIG. 2. illustrates a folded view of a device 200 for pregnancy device, according to an embodiment herein. The device 200 for pregnancy detection includes, a conical main body 202 including a first end 201 and a second end 202. The first end 201 includes a first opening 210 and the second end 202 includes a second opening 220 positioned opposite to the first opening 210.

In accordance with an embodiment of the disclosure, the device 100 further includes a first longitudinal crease 260 and a second longitudinal crease 270 opposite the first crease. In use, the first longitudinal crease 260 and the second longitudinal crease 270 are foldable and folded to have compact flattened packaging of the device 200.

In accordance with an embodiment of the disclosure, a perimeter of the first opening 210 is greater than a perimeter of the second opening 220. The device 200 is opened by applying simultaneous pressure to the first longitudinal crease 260 and the second longitudinal crease 270.

In accordance with an embodiment of the present disclosure, the device 200 further includes a pregnancy detection element 230 that is mounted at an edge of the second opening 220. In use, the pregnancy detection element 130 tests the urine and indicates a positive pregnancy result, or a negative pregnancy result, or a failed result. In operation, the pregnancy detection element 230 operates by immunologically detecting a plurality of morphological differences between human chorionic gonadotropin (hCG) and modified forms thereof, which are secreted into the body fluid (e.g. urine) of a pregnant female.

In accordance with an embodiment of the present disclosure, the first opening 201 of the first end 210 is configured to receive urine from a female user. In use, the first opening 201 of the first end 210 is configured to allow the female user to urinate while in standing position by slightly tilting (bending) her hips in a manner such that the device 200 is tilted downwards.

In accordance with an embodiment of the present disclosure, the device is made from a suitable material selected from a group including water-absorbent paper, cardboard, water dissolvable plastic-like film, and the like. In use, the device is biodegradable and environment friendly after being used by the female user.

In accordance with an embodiment of the present disclosure, the second opening 120/220 of the second end 102/202 is configured to discharge the urine from the female user after the urine has come in contact with the pregnancy detection element.

Those of ordinary skills in the art will appreciate that by way of alternate embodiments, the device 100 is capable of conducting one or more urine related tests as well, such as, for example, but not limited to, a UTI (Urinary Tract Infection) test, an ovulation or a fertility test, and the like. In use, such alternate embodiments shall include suitable replacements for the pregnancy detection element 130, as per the specific requirements.

In accordance with an embodiment of the present disclosure, a method for pregnancy detection includes the steps of: providing a pregnancy detection device to a female user; receiving urine from the female user into a first opening of a first end of the pregnancy detection device; flowing urine from the female user from the first opening towards a pregnancy detection element mounted at an edge of a second opening of a second end of the pregnancy detection device; testing and start indicating a positive pregnancy result, or a negative pregnancy result, or a failed result by the pregnancy detection element within 15 seconds of coming in contact with urine of the female user; and, discharging urine from the female user via the second opening after the urine has come in contact with the pregnancy detection element.

Therefore, as may be seen, various embodiments of the present disclosure as disclosed hereinabove provide significant advantages over prior art, such as, for example, but not limited to, providing a novel and inventive pregnancy test kit which can be used while standing and simply passing the liquid body waste in the product without coming into contact with the urine. Also, this present disclosure keeps the health care of all expecting woman in mind and is therefore very hygienic. Generally, embodiments of the instant disclosure will help the user to discard unhygienic process and provide the ease of use to test their pregnancy in coming era. The embodiments herein provide the great comfort to user to stand and test their pregnancy which none other product is providing in the market. The embodiments herein are also bio-degradable in nature, and, in simple words, it's a step ahead towards the ease of use

### EXAMPLES

### Example 1

Below is an exemplary non-limiting method of using the device of the invention:

Provider writes name and date of birth of patient on zippered bag containing the

Clean Screen Pregnancy Test funnel.

In the restroom, patient opens clear, zipped bag and extracts the funnel.

Patient unfolds funnel to its open position.

Patient holds the side handle and places funnel inside the toilet between their legs.

Patient urinates into and through funnel.

Urine encounters part of the circumferential hCG test strip located toward the bottom (small opening) of the funnel.

Patient extracts funnel from toilet.

Patient places funnel back into provided zippered bag and seals bag.

Urine is wicked down the test strip to encounter first the control reagent, and then the hCG reagent.

Provider reads test results through the clear zippered bag.

Provider disposes of the funnel and zippered bag together without coming in contact with the patient's urine.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

While there has been shown and described the preferred embodiment of the instant disclosure it is to be appreciated that the present disclosure may be embodied otherwise than is herein specifically shown and described and that, within said embodiment, certain changes may be made in the form and arrangement of the parts without departing from the underlying ideas or principles of this present disclosure as set forth in the Claims appended herewith. Therefore, the appended claims are to be construed to cover all equivalents falling within the true scope and spirit of the present disclosure.

What is claimed is:

1. A device comprising: a conical main body comprising a first end and a second end, wherein the first end comprises a first opening and the second opening comprising a narrower end positioned opposite to the first opening, wherein the conical body includes an extended portion for holding the device, and wherein the conical body includes a detection element to detect an analyte of interest, wherein the detection element is mounted closer to the second opening.

2. The conical device of claim 1, further comprising a first longitudinal crease and a second longitudinal crease, wherein the first longitudinal crease and the second longitudinal crease are capable of being folded to have a compact flattened effect.

3. The conical device of claim 1, wherein the first opening of the first end is configured to receive an analyte from a subject.

4. The conical device of claim 1, wherein when the analyte comes in contact with the detection element, the detection element detects the presence of analyte and indicates a positive or negative output if the analyte is present in the body fluid sample.

5. A device for a contactless pregnancy detection comprising a conical shaped main body including a first end having a first opening and a second narrower end having a second opening, a first longitudinal crease and a second longitudinal crease opposite the first crease allow the conical body to lay flat when not in use including a pregnancy detection element mounted closer to the second opening and a flap extending from the first end of the conical body to allow a user to hold the device.

6. A device for detection of human chorionic gonadotropin (hCG), the device comprising: a conical main body comprising a first end and a second end, wherein the first end comprises a first opening and the second end comprises a second opening positioned opposite to the first opening in a narrowing manner, a pregnancy detection element mounted proximate to the second opening; and a first longitudinal crease and a second longitudinal crease opposite the first crease, wherein the first longitudinal crease and the second longitudinal crease are capable of being folded to flatten the device, wherein the device is opened by applying simultaneous pressure to the first end of the first longitudinal crease and the second longitudinal crease, further comprising an extension proximate the first end to allow a subject to hold the device, wherein the first opening of the first end is configured to receive urine from a female user while the female user is in a standing position and enables the urine to pass through a pregnancy detection element proximate the second opening, and wherein when the urine comes in contact with the pregnancy detection element, the pregnancy detection element immunologically detects human chorionic gonadotropin (hCG) present in the urine of the female user and indicates a positive pregnancy result if the female user is pregnant.
